(19) United States
(12) Patent Application Publication
(10) Pub. No.: US 2026/0096824 A1
(43) Pub. Date: Apr. 9, 2026

(54) Bone Material Harvesting Device Having Radially Expandable And Retractable Shaft

(71) Applicant: Wright Medical Technology, Inc., Memphis, TN (US)

(72) Inventors: Richard Hales, Alpharetta, GA (US); Tushar Bhalla, Ghaziabad (IN); Letitia Tudor, Suwanee, GA (US); Stephanie Mansueti, Atlanta, GA (US); Basma Madid, Grenoble (FR)

(21) Appl. No.: 19/120,883

(22) PCT Filed: Oct. 13, 2023
(86) PCT No.: PCT/US2023/035103

§ 371 Date: Apr. 14, 2025

Related U.S. Application Data
(60) Provisional application No. 63/415,832, filed on Oct. 13, 2022.
(60) Provisional application No. 63/528,208, filed on Jul. 21, 2023.

(51) Int. Cl. A61B 17/16; A61B 17/00

(52) U.S. Cl. CPC A61B 17/1662; A61B 17/1635; A61B 17/164; A61B 2017/00473; A61B 2017/00477; A61B 2217/005

(57) ABSTRACT

A harvesting device includes an adaptor defining an internal cavity, a storage container coupled to the adaptor and in fluid communication with the internal cavity for receiving bone and/or cellular material extracted from a patient, and a reamer assembly. 2024/081400 The reamer assembly includes a shaft with a distal end arranged to receive a reamer head, an outer tube at least partially surrounding the shaft, and a deformable section. The deformable section has an elongated condition during a reaming procedure and a radially expanded condition for extracting the bone and/or cellular material from the patient. Thus, the reamer assembly is configured to mechanically extract bone and/or cellular material from the intramedullary canal of a patient.

### CROSS-REFERENCE TO RELATED APPLICATIONS

The present application claims the benefit of the filing date of U.S. Provisional Ser. No. 63/528,208 filed Jul. 21, 2023 and the benefit of the filing date of U.S. Provisional Ser. No. 63/415,832 filed on Oct. 13, 2022, the disclosures of which are hereby incorporated herein by reference.

### BACKGROUND OF THE INVENTION

The present invention relates to devices and methods for harvesting bone and/or cellular material for use in various medical treatments.

Pseudarthrosis, also referred to as non-union of bone, is a common complication of fracture treatment. Non-union occurs when a particular patient's fracture site fails to heal within a specified time and requires a surgical intervention to achieve proper union and mobility. In some cases, non-unions may be treated by bone grafting (e.g., allograft, autograft, or xenograft), through internal or external fixation, or a combination thereof. Bone grafting offers an opportunity to stimulate the fracture site so that bony formation occurs at the site to properly unionize the fracture.

Stem cells (e.g., Mesenchymal stem cells (MSCs), hematopoietic stem cells (HSCs), or other stem cells) are known to be useful with certain graft materials, or by themselves, to facilitate bone growth and formation when used appropriately. For example, adult MSCs are capable of differentiating into a variety of different cell types including osteoblasts (bone cells), chondrocytes (cartilage cells), and adipocytes (fat cells). As such, when incorporated with certain allograft material, stem cells can assist with the proper formation of bone and union of bone parts at a fracture site. Stem cells of the type discussed above must first be extracted from a patient and processed before use. As an example, extracted bone marrow of a patient can provide the necessary stem cells.

Bone marrow is typically extracted in a multi-staged procedure. In a first stage of the procedure, bone marrow is aspirated from a patient and then sent to a laboratory for processing. It is only then, in a second stage of the procedure, that the previously harvested bone marrow is processed for the patient's particular application. Bone marrow from the iliac crest is widely considered the “gold standard” for its superior biologic quality. The drawback of harvesting bone marrow from the iliac crest, however, is that the iliac crest naturally produces a limited quantity of marrow. For surgical interventions requiring a higher volume of bone marrow, such as trauma and orthopedic applications, bone marrow is often harvested from the medullary canal of a long bone (e.g., the femur or tibia), which naturally produces a higher volume of bone marrow than the iliac crest. Bone marrow harvested from the medullary canal has proven to be an effective alternative to bone marrow harvested from the iliac crest and is capable of providing significant regenerative potential.

In order to harvest the desired volume of marrow from the medullary canal, a surgeon must extract marrow located deep within the bone canal. This procedure is often performed using a harvesting system that is typically equipped with a reamer, an irrigation system, a suction source (OR suction) and a filter. Although these systems are generally sufficient in harvesting the desired bone material, they are aggressive and carry significant clinical risks such as severe blood loss that often necessitates blood transfusion treatments. In fact, while the medical community appreciates the regenerative potential of bone marrow harvested from the medullary canal of long bone, some medical professionals have begun to question whether harvesting the autograft materials is worth the significant clinical risks.

Known harvesting devices are also susceptible to clogging, which results in decreased aspiration function, especially while positioned deep within the medullary canal. Moreover, the harvested material is often diluted and/or contaminated by the water or other solution introduced by the irrigation system. Such dilution or contamination complicates the processing stage of the procedure and necessitates additional filtering or refining of the harvest.

### BRIEF SUMMARY OF THE INVENTION

In accordance with a first aspect of the present invention, a device for harvesting bone and/or cellular material (e.g., cancellous bone, bone chips, bone marrow, and stem cells) includes a deformable reamer assembly that mechanically extracts bone and/or cellular material with reduced, or completely without, aspiration. Among other advantages, the device is configured to perform clog-free transportation of even highly viscous material without the irrigation of fluids. As a result, the need for subsequent filtering and excessive processing is eliminated. Furthermore, the iterative reaming and mechanical extraction process reduces a surgeon's impulse to aggressively ream and aspirate bone and/cellular material which, in turn, reduces the likelihood of severe blood loss and the clinical complications associated with sever blood loss.

One embodiment of the harvesting device includes an adaptor defining an internal cavity, a storage container coupled to the adaptor and in fluid communication with the internal cavity for receiving bone and/or cellular material extracted from a patient, and a reamer assembly at least partially disposed within the adaptor. The reamer assembly includes a shaft defining a distal end arranged to receive a reamer head and an outer tube at least partially surrounding the shaft and having a deformable section, the deformable section having an elongated condition during a reaming procedure and a radially expanded condition for extracting the bone and/or cellular material from the patient.

The outer tube may be coupled to the shaft and movement of the outer tube relative to the shaft may transition the deformable section between the elongated condition and the radially expanded condition.

Movement of the outer tube relative to the shaft in a distal direction may transition the deformable section from the elongated condition to the radially expanded condition, and movement of the outer tube relative to the shaft in a proximal direction may transition the deformable section from the radially expanded condition to the elongated condition.

The reamer assembly may be retractable relative to the adaptor.

The deformable section may include a resilient material and the resilient material may be a biocompatible braided material.

The outer tube may include a handle provided with a locking mechanism arranged to secure the outer tube at a fixed position along a length of the shaft when the deformable section is in the elongated condition and/or in the radially expanded condition.

The adaptor may include an aspiration fitting arranged to securing a tubing, and the aspiration fitting may be arranged to rotate about a longitudinal axis of the adaptor and/or pivot about a connection point.

The adaptor may include a detachable protective sleeve with at least one protrusion on an exterior surface thereof.

In one aspect, a reamer assembly for extracting bone and/or cellular material includes a shaft defining a distal end arranged to receive a reamer head; and an outer tube at least partially surrounding the shaft and coupled to the shaft. The outer tube has a deformable section moveable between an elongated condition during reaming and a radially expanded condition for extracting the bone and/or cellular material from the patient.

The reamer assembly may further include the reamer head and the reamer head may be removably attachable to the distal end of the shaft.

The reamer head and the distal end of the shaft may be removably secured via a dove-tail type coupling.

The shaft may be permitted to rotate freely within the outer tube.

Movement of the outer tube relative to the shaft may transition the deformable section between the elongated condition and the radially expanded condition.

A cross-section of the deformable section may have a first diameter in the elongated condition and a second diameter in the radially expanded condition that is larger than the first diameter.

A method of collecting bone and/or cellular material during a harvesting procedure is also provided herein. The method includes the steps: (a) reaming a canal of a bone to generate bone and/or cellular material using a harvesting device including a reamer assembly having a deformable section; (b) deforming the deformable section of the reamer assembly; (c) retracting the reamer assembly to mechanically extract the bone and/or cellular material from the patient; and (d) collecting the bone and/or cellular material in a storage container.

The deforming step (b) may include moving an outer tube of the reamer assembly relative to a shaft of the reamer assembly.

The method may further include the steps: (e) removing a first reamer head from the reamer assembly after performing step (d); (f) securing a second reamer head to the reamer assembly after performing step (e), the second reamer head being larger than the first reamer head; and (g) reaming the canal of the bone to generate additional bone and/or cellular material using the harvesting device.

The bone may be one of a femur or a tibia bone.

The reaming step (a) may include reaming a counterbore within an entry portal of the bone, and the method may further include securing a protective sleeve of an adaptor within the reamed counterbore via an interference fit.

### BRIEF DESCRIPTION OF THE DRAWINGS

FIG. 1A is a perspective view of a harvesting device including a storage container, an adaptor, and a reamer assembly having a deformable section according to an embodiment of the invention.

FIG. 1B is an exploded view of the harvesting device of FIG. 1A.

FIG. 2A is a perspective view of the adaptor of FIG. 1 depicting a disconnectable protective sleeve and an aspiration fitting.

FIG. 2B is a perspective view of a tube connected to the aspiration fitting of the adaptor of FIG. 2B.

FIGS. 3A-3D are example protective sleeves of the adaptor of FIGS. 2A and 2B.

FIG. 3E is perspective view of an external stabilization device for stabilizing the protective sleeve of FIG. 2A within a reamed entry portal of bone.

FIG. 4A is a partial elevation view of the reamer assembly of FIG. 1 illustrating the deformable section in an undeformed, or elongated, condition.

FIG. 4B is a partial elevation view of the reamer assembly of FIG. 1 illustrating the deformable section in a deformed, or radially expanded, condition.

FIGS. 5A-5E are example locking devices for securing the deformable section of the reamer assembly of FIGS. 4A and 4B in the radially expanded condition.

FIG. 6 is a perspective view of the storage container of FIG. 1.

FIGS. 7A and 7B illustrate a medullary canal of a femur being reamed using the harvesting device of FIGS. 1A and 1B.

FIG. 7C illustrates a counterbore within the reamed femur.

FIGS. 8A and 8B illustrate a process of transitioning the deformable section of the reamer assembly of FIGS. 4A and 4B from the elongated condition to the radially expanded condition.

FIGS. 9A-9C illustrate a process of extracting bone and/or cellular material from the medullary canal of the femur using the harvesting device of FIGS. 1A and 1B.

FIG. 10 is a schematic representation illustrating a known harvesting device aspirating bone and/or cellular material from a medullary canal of a femur.

FIGS. 11A-21 illustrate alternative example deformable sections of the reamer assembly of FIGS. 4A and 4B.

### DETAILED DESCRIPTION

As used herein, the terms “proximal end” and “trailing end” refer to the end of the harvesting device, or a component thereof, nearest the user operating the device, and the terms “distal end” and “leading end” refer to the opposite end of the device furthest from the user. Also as used herein, the terms “substantially,” “generally,” “approximately,” and “about” are intended to mean that slight deviations from absolute are included within the scope of the term so modified.

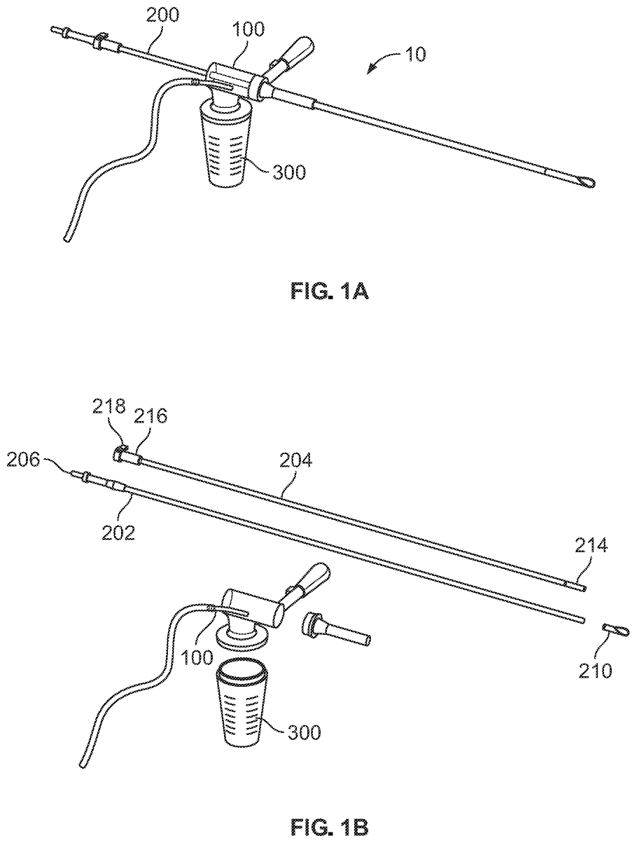

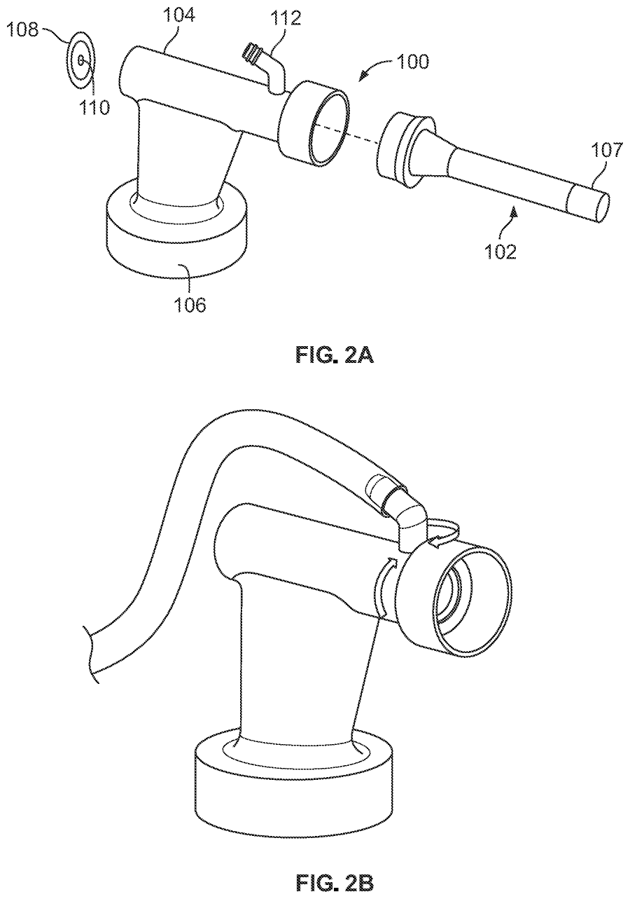

As shown in FIGS. 1A and 1B, harvesting device 10 includes an adaptor 100, a reamer assembly 200 for reaming and extracting bone and/or cellular material from a patient, and a storage container 300 for collecting the extracted material. The phrase bone and/or cellular material refers to material that is extractable from bone, which may optionally then be processed and/or separated to produce another material. For instance, bone and/or cellular material may include cancellous bone, cortical bone (in the form of chips or morselized bone), bone marrow, or stem cells produced from any of the foregoing materials. Such materials are frequently found in the iliac crest, or the medullary canal of a long bone, as well as the bone surrounding the medullary canal. Harvesting device 10 may be formed, in part, from a transparent medical grade glass or plastic, thereby permitting an operator using the device to observe the collection of bone and/or cellular material during a harvesting procedure.

Adaptor 100 defines an internal cavity that communicatively couples reamer assembly 200 and storage container 300. With additional reference to FIGS. 2A and 2B, adaptor 100 includes a distal portion in the form of a protective sleeve 102, a proximal portion 104 and a connector 106. Connector 106 may include internal threading (not shown) for removably securing storage container 300 to adaptor 100, and can be provided at any suitable location along the length of the adaptor, for example, between the distal and proximal portions. Alternatively, storage container 300 may be coupled to adaptor 100 via an interference or snap-fit connection, or any other connector known in the art.

The protective sleeve 102 of adaptor 100 may be formed as a hollow tube with a rounded distal end that protects surrounding tissue and reduces trauma when reamer assembly 200 is operated within the medullary canal of bone. The distal portion of protective sleeve 102 may be tapered and designed to sit at least partially within an entry portal of a reamed bore to form a seal with the bone and prevent extracted bone and/or cellular material from leaking around the protective sleeve regardless of local bone topography. An exterior surface of the tapered portion may include one or more protrusions 107 for securely stabilizing the tapered portion of protective sleeve 102 within the entry portal of the bone via an interference fit. The combination of the tapered distal portion of protective sleeve 102 and one or more protrusions 107 may alleviate the need for an operator to physically press the distal end of the protective sleeve against a surface of bone, around the bore, during the reaming and harvesting steps, thus freeing the operator's hands to operate reamer assembly 200.

An end cap 108 may be securable to the proximal end of proximal portion 104. End cap 108 can be manufactured from an elastic polymeric material such as rubber and can include a centrally located slit 110 or other suitable opening that allows reamer assembly 200 to slide relative to and through adaptor 100. Slit 110 may be sized to form a seal with reamer assembly 200. As a result, when reamer assembly 200 is retracted through slit 110, end cap 108 prevents bone and/or cellular material from passing through the proximal end of adaptor 100.

Adaptor 100 may optionally include an aspiration fitting 112 arranged to secure tubing as shown in FIG. 2B. During a procedure, tubing may be connected between a suction source (not shown) and aspiration fitting 112 to provide a negative pressure within adaptor 100. The negative pressure within adaptor 100 is designed to guide bone and/or cellular material into the distal end of protective sleeve 102 after it has been extracted from the intramedullary canal by reamer assembly 200. As a result, the suction force applied to the intramedullary canal by harvesting device 10 may be much lower than conventional harvesting devices that rely solely on aspiration to extract bone and/or cellular material from an intramedullary canal. Therefore, even in situation in which adaptor 100 includes aspiration fitting 112, harvesting device 10 can drastically reduce, if not eliminate, the risk of serious blood loss during a harvesting procedure.

Aspiration fitting 112 may be rotatably secured to a ring configured to rotate about a longitudinal axis of adaptor 100. In one example, the ring may be adapted to rotate approximately 120° about the longitudinal axis of the adaptor to move aspiration fitting 112 from the upper side of proximal portion 104 to either lateral side of the proximal portion. Furthermore, aspiration fitting 112 may be pivotably connected to proximal portion 104 such that the aspiration fitting is able to pivot 360° about its connection point. The rotatability and pivotability of aspiration fitting 112 can allow an operator to reposition the tubing as desired during an operation so that the tubing does not interfere with the procedure.

In some implementations, protective sleeve 102 and proximal portion 104 may be integrally formed as a single unitary component. Alternatively, as shown in FIG. 2A, protective sleeve 102 may be removably connectable to proximal portion 104. When protective sleeve 102 is removably connectable to proximal portion 104, the proximal end of the protective sleeve includes a coupling mechanism for removably securing the protective sleeve to proximal portion 104. The coupling mechanism may include threading, an interference fit, a twist-and-lock connection, or any other connection-disconnection mechanism known in the art. One advantage of utilizing a disconnectable protective sleeve 102 is the ability to interchange between protective sleeves of different geometries based upon operational considerations including, but not limited to, the size of the reamer and the bone and/or the entry portal location of the procedure.

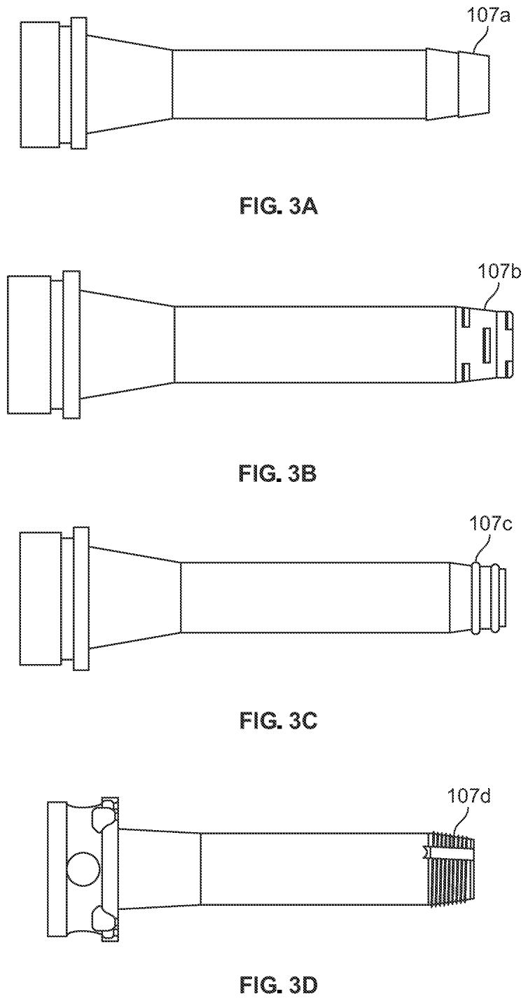

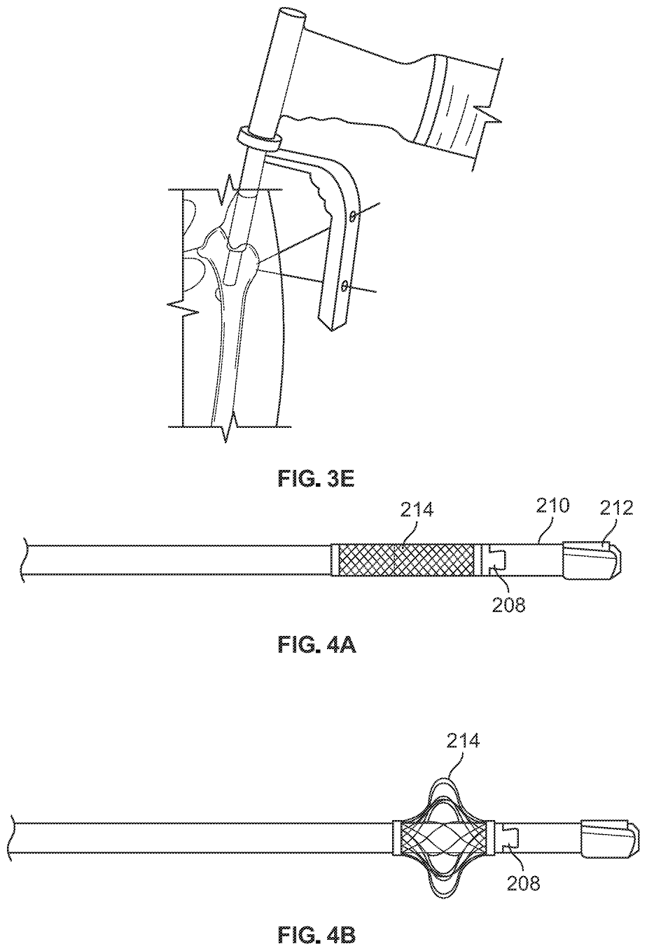

FIGS. 3A-3D illustrate example protective sleeves 102 that may be interchangeably connected to proximal portion 104. FIG. 3A includes a protrusion 107a in the form of a barbed distal end in which the barb circumscribes the tapered portion. FIG. 3B includes a series of protrusions 107b in the form of a plurality of discontinuous ridges circumscribing the tapered portion. FIG. 3C includes a series of protrusions 107c in the form of a plurality of continuous ridges circumscribing the tapered portion. FIG. 3D includes a series of protrusions 107d in the form of a self-tapping helical thread that may be manually rotated and secured into bone. FIGS. 3A-3D are merely illustrative examples. The one or more protrusions 107 may have any other structure that secures protective sleeve 102 within an entry portal of bone and that provides a seamless transition between the intramedullary canal and an inner diameter of the protective sleeve.

Although the foregoing description has focused on securing protective sleeve 102 within an entry portal of a reamed bone, it is also contemplated that the protective sleeve may alternatively be secured against an exterior surface of the bone, around a reamed bore, using an external fixation device 114, thereby stabilizing the protective sleeve and freeing the operator's hands to operate reamer assembly 200. FIG. 3E depicts an example external fixation device 114 that utilizes pins for securing protective sleeve 102 against a surface of a reamed femur.

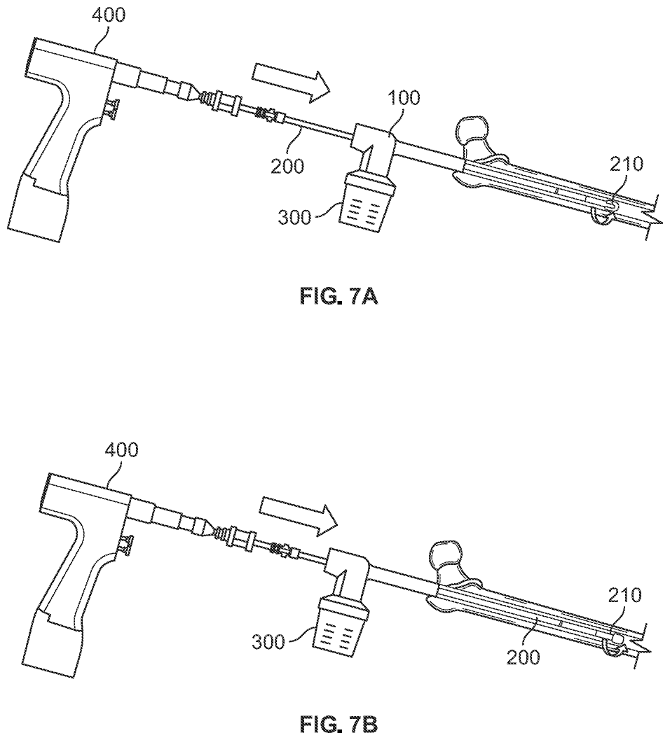

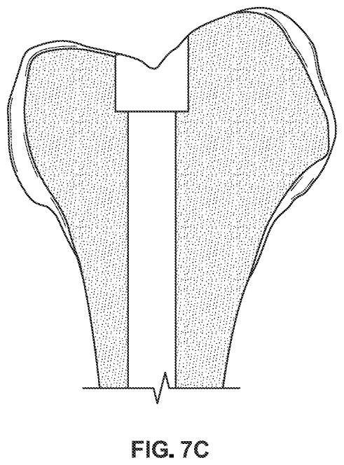

Reamer assembly 200, as shown in FIGS. 1A, 1B, 4A and 4B, includes a shaft 202 and an outer tube 204. Shaft 202 and outer tube 204 may be formed of a resilient and flexible material, such as carbon fiber, that allows the reamer assembly 200 to bend as the reamer assembly navigates through the medullary canal of a long bone. The proximal end of shaft 202 includes a connection 206 designed to engage with a powered or manual instrument 400 (FIGS. 7A-7B) for rotating the shaft during a reaming procedure. Connection 206 may be keyed for insertion into a likewise keyed portion of instrument 400. In one example, connection 206 may have a hexagon shaped cross-section.

A coupling device 208 secures a reamer head 210 to the leading end of shaft 202. The coupling device may be a dove-tail type coupling device having interlocking male and female components as described in U.S. Pat. No. 5,203,595 which is hereby incorporated by reference herein. For example, the distal end of shaft 202 may include an enlarged male component that interlocks with a corresponding female component provided on the trailing end of reamer head 210. As a result, coupling device 208 allows a user to exchange one reamer head 210 for another reamer head while preventing the secured reamer head from unintentionally being disconnected from the shaft during a reaming procedure. Each reamer head 210 may be cannulated to receive K-wire and include a cutting tip 212 for reaming bone.

The leading end of outer tube 204 may be attached to coupling device 208 via a rotary joint that permits shaft 202 to be rotated within the outer tube and that prevents the outer tube from being retracted independently of the shaft. With specific reference to FIGS. 4A and 4B, outer tube 204 may include a deformable section 214 capable of transitioning between an elongated condition (FIG. 4A) during reaming and a radially expanded condition (FIG. 4B) for extracting bone and/or cellular material from a patient. Deformable section 214 may be formed of a biocompatible and resilient material, for example, a metal or metal alloy such as nitinol. Thus, when outer tube 204 is moved distally relative to shaft 202, the braided material of deformable section 214 compresses in a longitudinal direction and expands in a radial direction as shown in FIG. 4B. The nitinol may be covered with a thin silicone rubber, another elastomer, or a fabric to aid in extracting bone and/or cellular material from a patient during a procedure. Although the braided material illustrated in FIGS. 4A and 4B has a “natural” elongated condition, it will be understood that the braided material may alternatively have a natural, or resting state, in which the material is in the radially expanded condition. In such scenarios, an operator may retract outer tube 204 in a proximal direction relative to shaft 202 to transition deformable section 214 from radially expanded condition to the elongated condition.

It will also be understood that deformable section 214 may be formed from other materials and in other manners so long as the deformable section can be transitioned between an elongated condition during reaming and a deformed, or radially expanded, condition for extracting bone and/or cellular material. Thus, when reamer assembly 200 is retracted through adaptor 100 with deformable section 214 in the radially expanded condition, the reamer assembly will mechanically drag reamed bone and/or cellular material located proximally of the deformable section into adaptor 100. Various constructions of alternative deformable sections are discussed hereinafter.

As shown in FIGS. 1A and 1B, outer tube 204 may include a handle 216 that assists the user in moving the outer tube in a distal direction relative to shaft 202 to transition the deformable section between the elongated condition and the radially expanded condition, and a locking device 218 can be provided on the handle. More specifically, locking device 218 can be designed to secure outer tube 204 at one or more locations along the length of the shaft which, in turn, locks deformable section 214 in the elongated condition and/or at one more expanded diameters (e.g., partially expanded, fully expanded). The deformable section can thus be expanded to approximately match the diameter of differently sized bores and locked at such diameter.

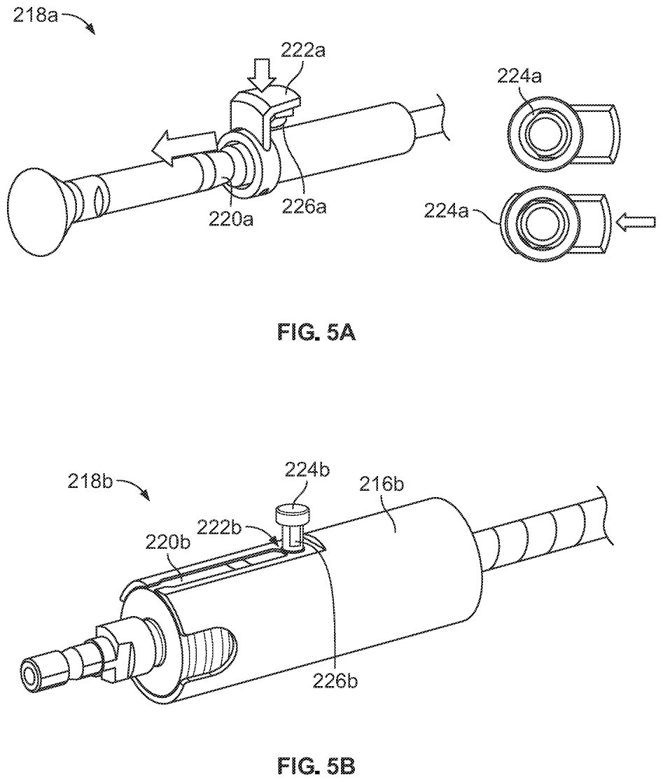

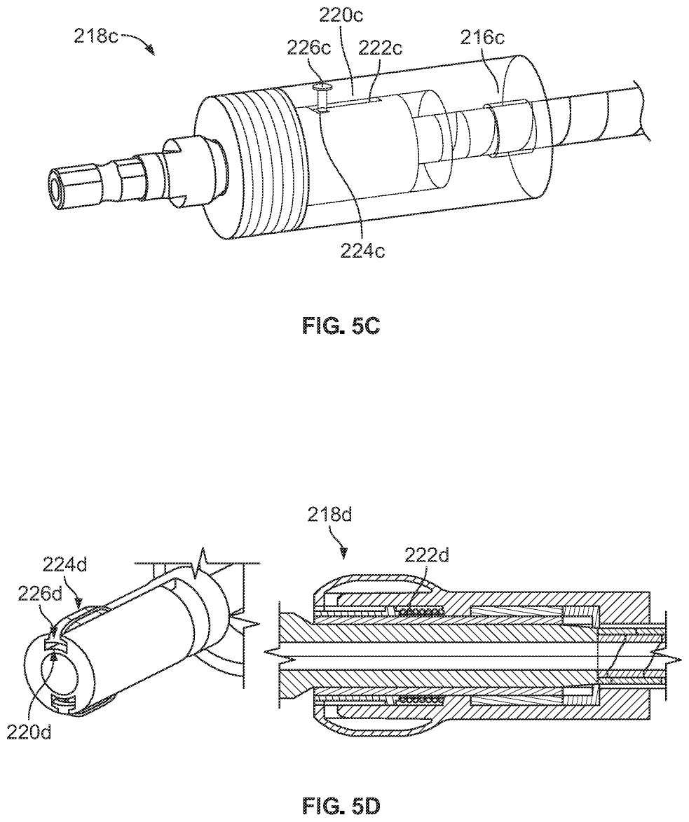

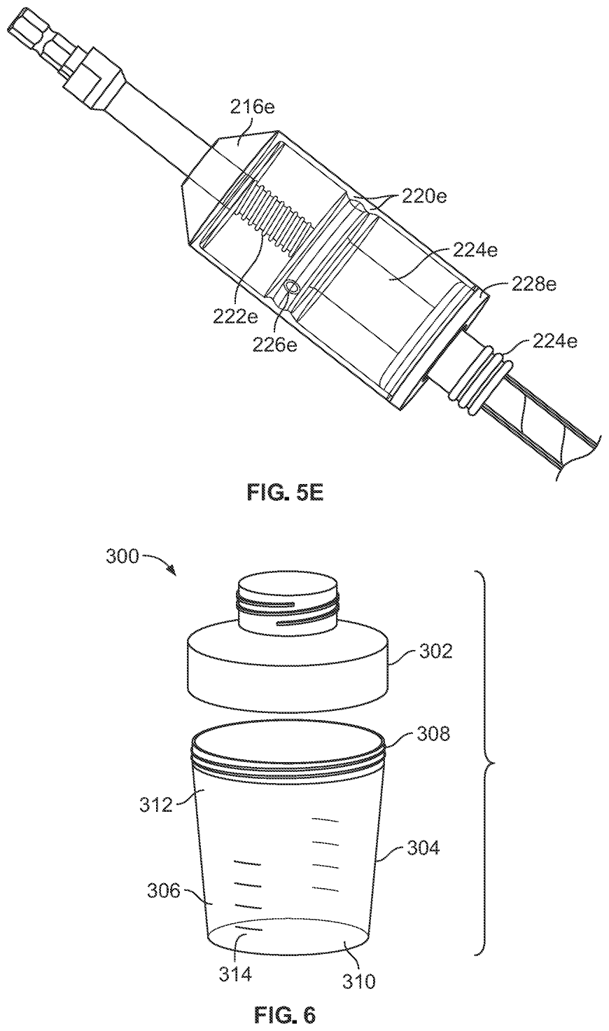

FIGS. 5A-5E illustrate example locking devices 218. With reference to FIG. 5A, shaft 202 includes one or more annular grooves 220a (or alternatively ridges) extending about the shaft. The locking mechanism 218a includes a button 222a, a clip 224a disposed about the shaft 202, and a spring 226a that biases the clip into contact with the shaft. Thus, when locking mechanism is in the elongated condition, clip 224a is biased into one of the annular grooves 220a of shaft 202 such that outer tube 204 cannot move along the length of the shaft. When an operator desires to expand or compress deformable section 214, the operator may press button 222a which, in turn, compresses spring 226a and moves clip 224a away from shaft 202 and out from within an annular groove 220a. Once clip 224a has cleared annular groove 220a, the operator may freely slide outer tube 204 relative to the shaft in the proximal or distal direction to expand or compress the deformable section as desired. After deformable section 214 has been transitioned to a desired condition, the operator may release button 222a, allowing spring 226a to again bias clip 224a within another one of the annular grooves 220a and lock outer tube 204 relative to shaft 202.

FIG. 5B illustrates another example locking device 218b. Handle 216b includes a longitudinal track 220b provided with one or more narrowings 222b. Locking device 218b includes a spring-loaded button 224b extending from an interior of handle 216b to an exterior of the handle. Spring-loaded button 224b is provided with a widened base 226b that has a dimension greater than narrowings 222b. The spring biases widened base 226b to an exterior of handle 216b such that the cooperation between the narrowings 222b and the widened base 226b of spring-loaded button 224b prevents handle 216b from sliding relative to shaft 202. To move handle 216b, the operator may press spring-loaded button 224b, thereby pressing widened base 226b within an interior of outer tube 204, and allowing the smaller dimensioned upper end of the spring-loaded button to pass through the narrowing 222b as the operator slides the handle along the length of shaft 202.

FIG. 5C illustrates yet another example locking device 218c. Shaft 202 includes a track 220c including a longitudinal track 222c and one or more annular branches 224c extending from the longitudinal track 222c. Handle 216c has an internal pin 226c disposed within track 220c. To move outer tube 204 relative to shaft 202, the operator may rotate handle 216c relative to the shaft to position pin 226c within longitudinal track 222c. Once pin 226c has been positioned within longitudinal track 222c, outer tube 204 is freely slidable along the length of shaft 202. To lock the position of outer tube 204 relative to shaft 202, the operator may again rotate handle 216c to position pin 226c within one of the annular branches 224c.

FIG. 5D illustrates still another example locking device 218d. As shown in FIG. 5D, shaft 202 may include diametrically opposed cutouts 220d. Handle 216d may include a spring 222d and diametrically opposed flanges 224d, each of which are provided with an end tab 226d arranged to be secured within the cutouts 220d of shaft 202. To compress the deformable section 214, and secure the deformable section in the compressed condition, the operator may grip flanges 224d and pull outer tube 204 in the proximal direction to compress spring 222d and position end tabs 226d within the cutouts 220d of shaft 202. To release locking device 218d and return deformable section 214 to an expanded condition, the operator may further retract outer tube 204 in the proximal direction while pulling flanges 224d apart from each other such that ends tabs 226d are released from cutouts 220d.

FIG. 5E illustrates yet another example locking device 218e. The handle 216e of locking mechanism 218a includes an insert 224e containing one or more plunger balls 226e, a spring 222e, and a stopper 228e. When spring 222e is in a natural condition, the plunger balls 226e are biased on the ridges of handle 216e such that outer tube 204 cannot move along the length of shaft 202. When an operator desires to expand or compress deformable section 214, the operator may pull on distal rings of the insert 224e, which in turn compresses the plunger balls 226e and moves insert 224e out of the annular ridge 220e. Once insert 224e has cleared the annular ridge, the operator may freely slide outer tube 204 relative to shaft 202 in the proximal or distal direction to expand or compress the deformable section as desired. After deformable section 214 has been transitioned to a desired condition, the operator may release insert 224e, allowing spring 222e to compress the insert 224e.

Locking devices 218a-218d depicted in FIGS. 5A-5E merely illustrate example locking devices that may be used to lock outer tube 204 relative to shaft 202. However, other suitable locking devices configured to achieve the same purpose may be utilized.

Turning now to FIG. 6, storage container 300 can include a lid 302 and a collection canister 304. Lid 302 may include external threads for securing storage container 300 to connector 106 and internal threads (not shown) for securing lid 302 to collection canister 304. In other examples, lid 302 may be integrally formed with adaptor 100.

Collection canister 304 can be formed from a medical grade plastic or glass and includes a cylindrical sidewall 306 that extends from a top end 308 to an annular base 310. The top end 308 of collection canister 304 can be provided with external threads 312 to threading securing lid 302 to collection canister 304. The sidewall 306 may be ergonomically shaped to aid a user in connecting and disconnecting the storage container 300 to the adaptor 100 and for gripping the collection canister 304 during a harvesting operation. In some embodiments, an indicator scale 314 may be molded, or otherwise imprinted on, collection canister 304 to assist a user in quickly determining the volume of bone and/or cellular material that is present within the collection canister.

Harvesting device 10 may be used to harvest bone and/or cellular material in a harvesting method as provided herein. The method can generally include extracting bone and/or cellular material from a patient and then sending it to a storage, separation and processing facility (e.g., a “biobank”) for use in a later surgical procedure involving that patient, or a different patient. Bone and/or cellular material can be safely extracted from the patient, using harvesting device 10, for subsequent processing and reuse without placing the patient at risk.

Referring to FIGS. 7A-9C, a method of extracting bone and/or cellular material from a patient is described, using an IM nail procedure remedying a patient's fractured femur as an illustrative example. It will be understood, however, that harvesting device 10 significantly reduces surgical complications when harvesting bone and/or cellular material within a medullary cavity and, thus, may likewise be used to harvest material from within the medullary canal of the tibia or any other bone from which bone and/or cellular material may be harvested.

In reducing a fractured femur, a surgeon may first approach the fracture by making an incision in the patient's skin adjacent the hip. The surgeon may then resect the greater trochanter, thereby creating an opening to the medullary canal of the femur. Resection of the greater trochanter generates loose cortical and cancellous bone which may optionally be collected, for example, by hand.

A K-wire may then be introduced through the patient's skin and into the medullary canal of the femur. The surgeon may then secure instrument 400 to the connection 206 of reamer assembly 200 and use the K-wire to guide harvesting device 10 through an entry point and into the medullary canal of the femur. With the K-wire extending within the medullary canal and past the fracture site, instrument 400 may be used to rotate shaft 202 relative to outer tube 204, as shown in FIGS. 7A and 7B, to bore through cortical and cancellous bone, as well as bone marrow of the patient, thereby generating bone and/or cellular material in the medullary canal.

In situation in which the surgeon deems it appropriate to use a counterbore reamer, a stepped or “counterbored” opening can be formed in the bone adjacent to the entry point as shown in FIG. 7C. The surgeon may then press the tapered portion of protective sleeve 102 within the counterbore and secure the one or more protrusions 107 of the protective sleeve within the counterbore to stabilize the protective sleeve therein via a press-fit connection.

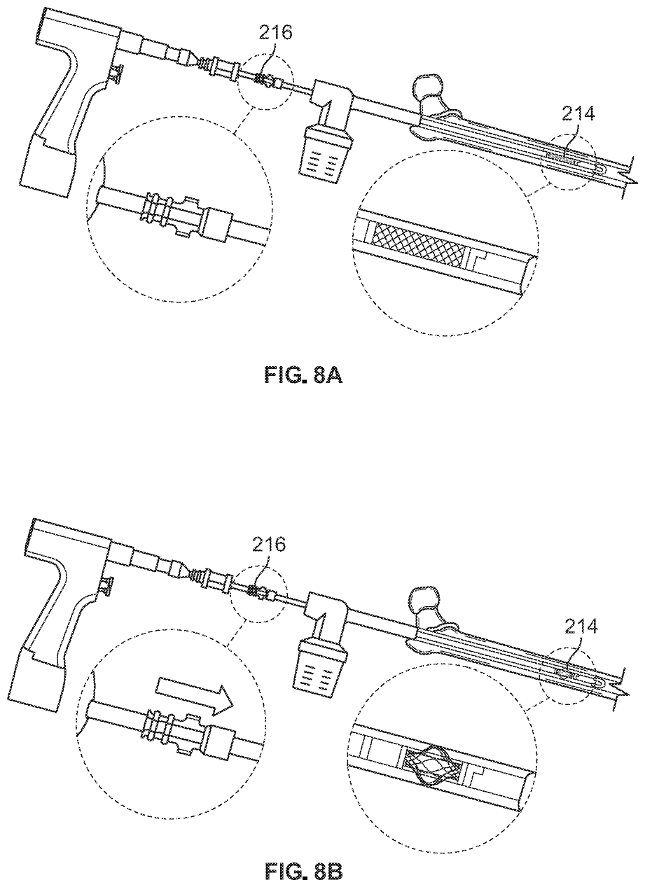

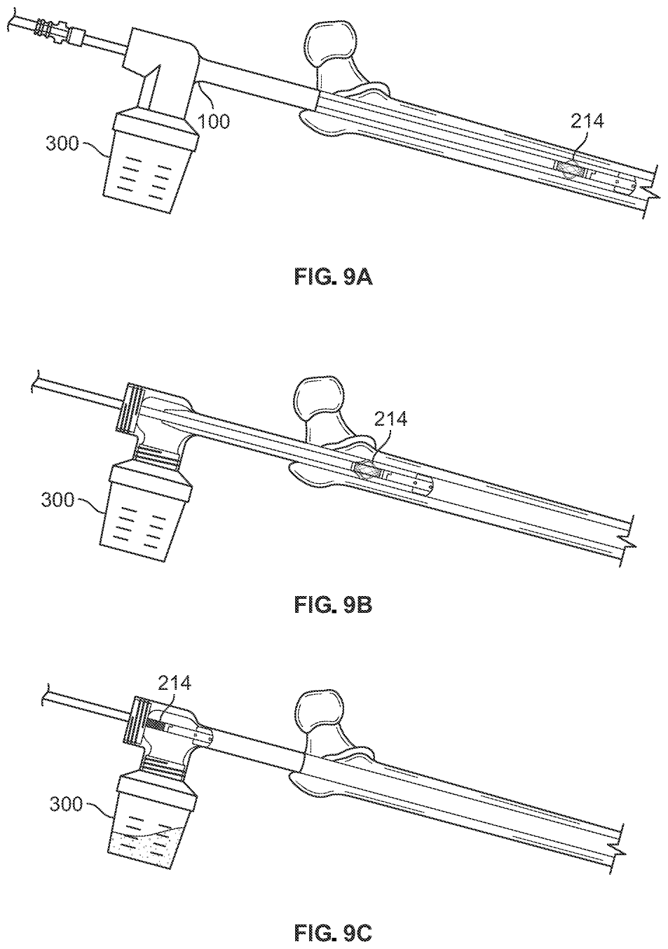

With the hands of the surgeon free, the surgeon may then grasp handle 216 and move outer tube 204 toward a leading end of reamer assembly 200 as shown in FIGS. 8A-8B. Moving outer tube 204 in this direction will cause the braided material of deformable section 214 to transition from the elongated condition to the radially expanded condition and to engage the wall of the bored medullary canal. With the deformable section 214 in the expanded condition, the surgeon may actuate locking device 218 to secure the deformable section in the expanded condition and then retract reamer assembly 200. As depicted in FIGS. 9A-9C, retraction of reamer assembly 200 will mechanically drag the reamed bone and/or cellular material into adaptor 100 as the deformable section is retracted. Gravity can then cause the bone and/or cellular material to fall into storage container 300. It will be appreciated that when protective sleeve 102 is stabilized within the reamed counterbore and around the bore, bone and/or cellular material should not leak around the protective sleeve. Moreover, in situations where the surgeon deems it appropriate, aspiration may also be used to guide extracted bone and/or cellular material into the distal end of protective sleeve 102.

Depending on the size of the IM implant, the surgeon may need to replace reamer head 210 with another reamer head of a different size or type. For example, if a larger bore needs to be formed in the intramedullary canal, the surgeon may disconnect reamer head 210 from shaft 202 by decoupling the male and female components of coupling device 208 before securing a larger reamer head to the shaft. With the larger reamer head secured to shaft 202, the procedure described above with respect to FIGS. 6A-8C may be repeated. The surgeon may optionally also replace the protective sleeve 102 in view of the new reamer head 210 to adjust the geometry of the protective sleeve or the structure of the one or more protrusions. This iterative process of reaming bone with a new reamer head and extracting bone and/or cellular material from the medullary canal of the patient may be repeated until the surgeon is satisfied that a bore of sufficient size has been created to receive the IM implant.

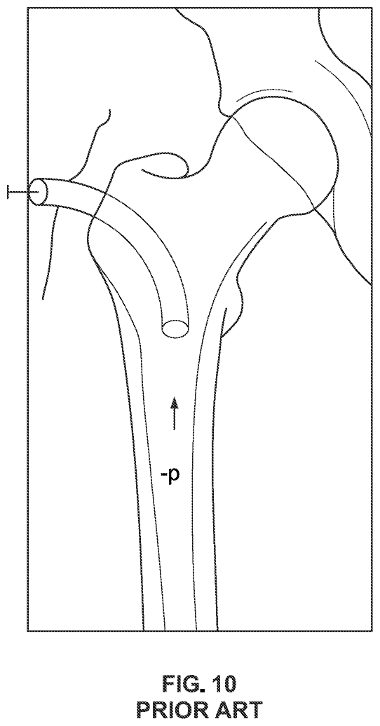

FIG. 10, illustrates a known harvesting device designed to extract bone and/or cellular material via a reaming and aspiration process. As shown in FIG. 10, the medullary canal is susceptible to becoming negatively pressurized during aspiration. The negative pressure can result in the aspiration of blood and can result in sever blood loss. Additionally, as the medullary canal drops in pressure, the aspiration power of the assembly is reduced, resulting in decreased aspiration efficiency often clogging the assembly.

In contrast, harvesting device 10 offers several advantages over the known harvesting device depicted in FIG. 10. For example, deformable section 214 may mechanically extract bone and/or cellular material without aspiration or irrigation of fluids. Harvesting device 10 thus can be safer for the patient and capable of extracting even highly viscous bone and/or cellular material without diluting the bone and/or cellular material with fluids. As a result, the need for subsequent filtering and excessive processing may be reduced if not eliminated. Moreover, the iterative process of reaming bone, mechanically extracting the material, and exchanging the reamer head 210 for a slightly larger reamer head, can reduce a surgeon's impulse to aggressively ream and aspirate the bone and/or cellular material which, in turn, can reduce the likelihood of severe blood loss and the clinical complications associated with same.

After the bone and/or cellular material has been harvested, the surgeon may implant the IM nail and finish the IM nail procedure. The bone and/or cellular material may then be sent to a biobank as separate collections, or as a cumulate harvest, for subsequent processing and use in a later surgical procedure involving that patient, or a different patient.

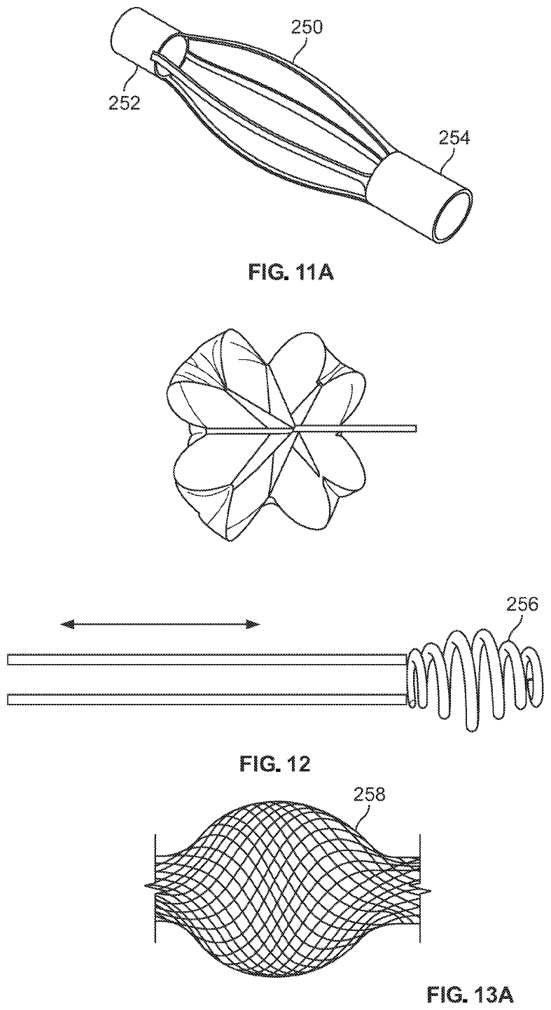

FIGS. 11-21, illustrate example deformable sections 214. Turning now to FIG. 11, deformable section 214 may be formed of a plurality of arms 250 extending between a proximal hub 252 connected to a distal end of outer tube 204 and a distal hub 254 connected to coupling device 208. In this regard, as outer tube 204 is telescoped over shaft 202 in the distal direction, arms 250 will expand from the elongated condition to the expanded condition. Arms 250 may be formed from nitinol, or any other resilient material, and may be covered with a thin layer of silicone, or any other elastomer. Alternatively, as shown in FIG. 12, deformable section 214 may be formed as a barrel spring 256 connected to the distal end of outer tube 204 and coupling device 208. The barrel spring 256 may be elongated in a resting state. Consequently, as outer tube 204 is telescoped over shaft 202 in a distal direction, barrel spring 256 may be transitioned to the radially expanded condition.

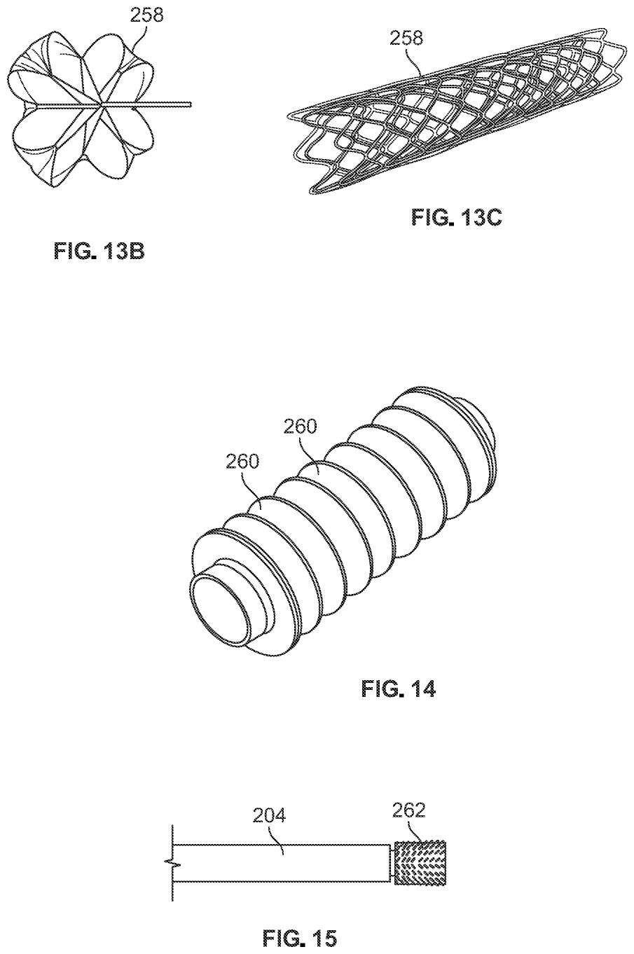

Turning to FIGS. 13A-13C, deformable section 214 may be formed of a braided resilient metal or metal alloy such as nitinol. As shown in FIG. 13A, the braided material 258 may be generally spherical or cylindrical in shape when in the expanded condition. In another example, braided material 258 may be shaped generally as an umbrella or a parachute when in the expanded condition as shown in FIG. 13B. Additionally, or alternatively, the braided material may be constructed from a plurality of superimposed braided walls, thereby improving the structural integrally of deformable section 214 as shown in FIG. 13C. The braided materials may be formed of the same material or of different materials. In one example, when the braided materials are formed from different materials, the inner braided material may be selected from a material having a relatively higher mechanical strength to improve the structural integrity of deformable section 214 and the outer braid may be selected from a material that has a relatively lower mechanical strength that may be efficiently transitioned between the elongated condition and the radially expanded condition and that also conforms to the topography of the reamed intramedullary canal.

FIG. 14 depicts a deformable section 214 formed of an elastomer such as rubber defining a plurality of bellows 260. In this regard, deformable section 214 may be stretched to the elongated condition when the outer tube 204 is retracted in the proximal direction and radially expanded when the outer tube is telescoped over shaft 202 in the distal direction.

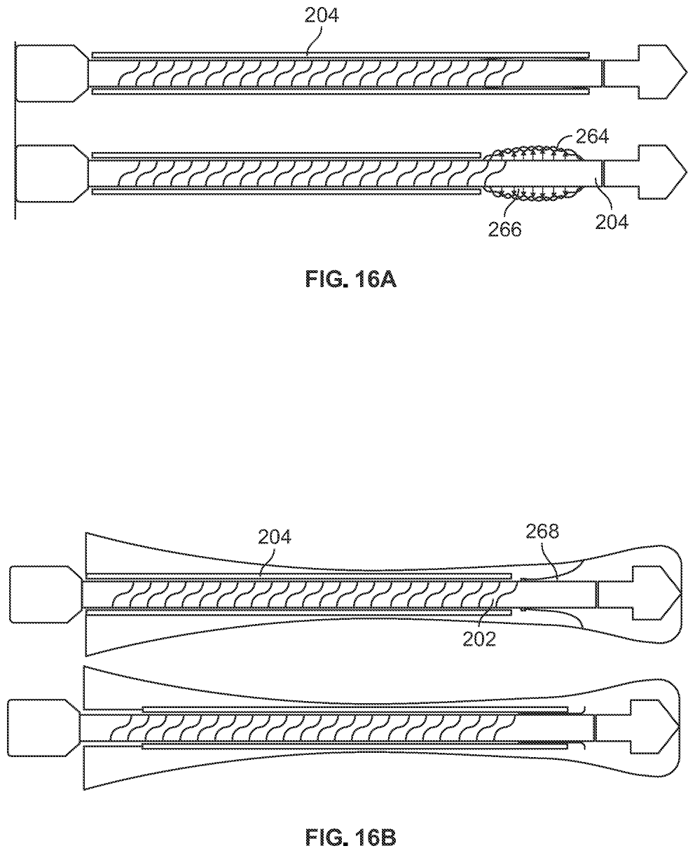

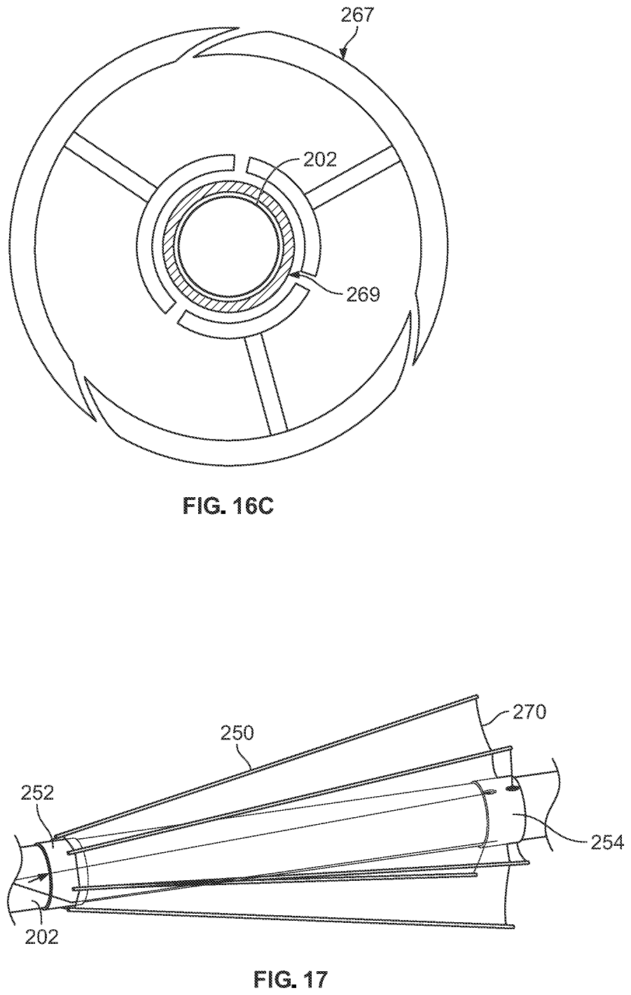

In alternative constructions, deformable section 214 may be disposed on shaft 202. As shown in FIG. 15 deformable section 214 may be formed as a series of brush-like fibers 262 covered be an elastomer or fabric material (not shown). The brush-like fibers 262 may be expanded to the radially expanded condition when the outer tube 204 is retracted in the proximal direction to un-sheath the brush-like fibers. On the other hand, brush-like fibers 262 may be collapsed when the outer tube 204 is extended distally over the brush-like fibers to collapse the fibers. As shown in FIG. 16A, deformable section 214 may be formed from a radially expandable elastomer 264 attached to shaft 202 at a first end and attached the shaft at a second end. The elastomer 264 may be expanded to the radially expanded condition when the other tube 204 is retracted in the proximal direction to un-sheath the elastomer and collapsed to the elongated condition when the outer tube is extended distally over the elastomer. In some implementations, one or more springs 266, or linkages, may be disposed between shaft 202 and elastomer 264 to assist the elastomer is expanding in the radial direction after the elastomer has been un-sheathed. FIG. 16B illustrates a similar deformable section formed as a cone 268 having a first end attached to shaft 202 and an opposite free end (e.g., not attached to shaft 202). Thus, when outer tube 204 is retracted in the proximal direction, the free end of cone 268 may be expanded about shaft 202. On the other hand, when outer tube 204 is telescoped distally over shaft 202, cone 268 may be collapsed in a radially direction toward shaft 202 and within the outer tube.

FIG. 16C illustrates yet another deformable section in the form of a split ring 267 having spring-like spokes disposed about a tapered tubing 269 which, in turn, is disposed about shaft 202. Tapered tubing 269 tapers from a proximal end having a larger cross-section to a distal end having a smaller cross-section. In this regard, as tapered tubing 269 is extended in a distal direction about shaft 202, the larger proximal portion of the tapered tubing pushes the spring like spokes outwardly to expand split ring 267. If the operator desires to collapse split ring 267, the operator may retract tapered tubing 269 such that the smaller cross-section of the tapered tubing is disposed within split ring 267 thus the spring-like spokes to return to their natural condition and collapse the split ring radially inward.

FIG. 17 illustrates still another example deformable section 214 disposed on shaft 202. More particularly, deformable section 214 includes a plurality of arms 250, a proximal hub 252 slidably disposed on shaft 202, and a distal hub 254. Each arm 250 has first end attached to proximal hub 252 and a second end defining an aperture. Arms 250 may be formed of a metal or a metal alloy having a naturally expanded condition. Put differently, when outer tube 204 is retracted in a proximal direction to un-sheath deformable section 214, arms 250 may be radially expanded. A wire 270 may be threaded through the aperture of each arm, an aperture of a distal hub 254, and then connected to proximal hub 252. As a result, an operator may slide proximal hub 252 about shaft 202 to transition arms 250 between the elongated condition and the radially expanded condition independently of outer tube 204. More particularly, with outer tube 204 in a retracted state, proximal hub 252 may be slid in the proximal direction to tension wires 270 and pull the second end of arms 250 radially inward towards shaft 202. On the other hand, an operator may slide proximal hub 252 in the distal direction to add slack to wires 270 and allow arms 250 to radially expand outwards to its natural condition.

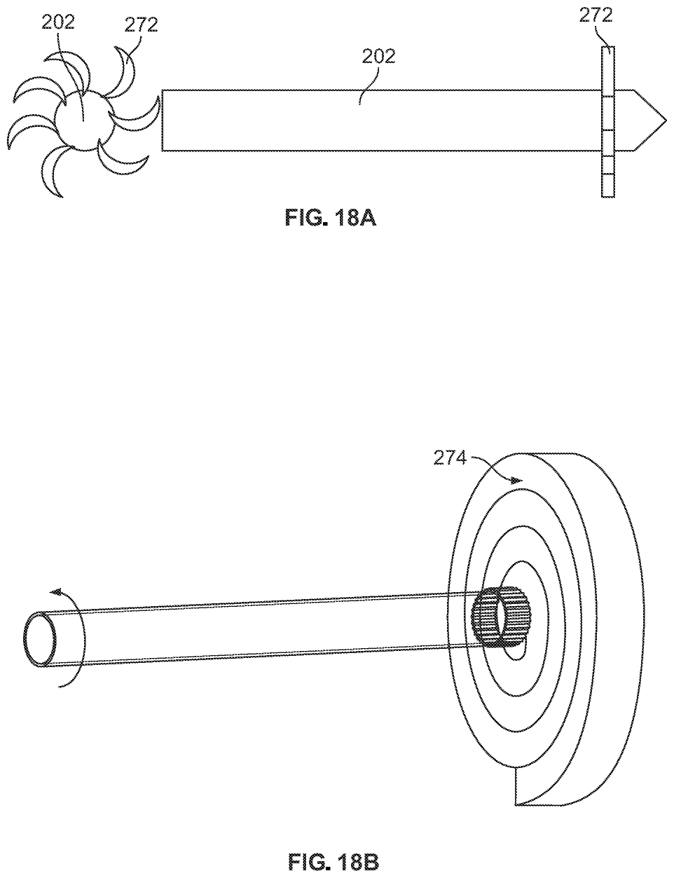

Turning to FIG. 18A, deformable section 214 may be formed of a plurality of blades 272 (or hooks) formed or a silicone or rubber material and disposed on shaft 202 to be actuatable between a collapsed condition and a radially expanded condition when the shaft is rotated. For example, blades 272 may be actuated to expand when shaft 202 is rotated in a counter-clockwise direction and collapsed when the shaft is rotated in a clockwise direction. In this regard, blades 272 remain in a collapsed condition during a reaming procedure when shaft 202 is rotated in a clockwise direction. After reaming, the operator may then rotate shaft 202 in the counter-clockwise direction to expand blades 272 the shaft is pulled in a proximal direction to extract bone and/or cellular material from the intramedullary canal of the patient. Alternatively, blades 272 may be actuated via a trigger (not shown) disposed on a proximal portion of reamer assembly 200. FIG. 18B illustrates a similar deformable section that replaces blades 272 with a spiral spring 274 covered with a fabric. Shaft 202 may be coupled to a center portion of spiral spring 274 via a gear. Spiral spring 274 is actuated in the same manner as blades 272. That is, spiral spring may be retained in the radially collapsed condition when shaft 202 is rotated in a clockwise direction during a reaming procedure and then may be rotated in a counter-clockwise direction to radially expand the spiral spring.

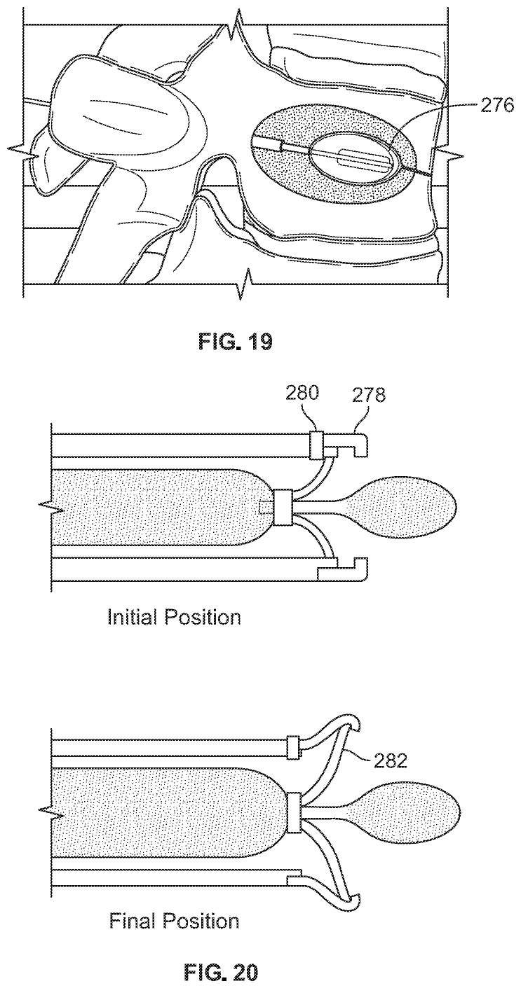

FIG. 19 illustrates yet another example of deformable section 214. As shown in FIG. 19, deformable section 214 may include one or more balloons 276. Shaft 202 may define a tubing extending through a sidewall thereof between an inflation port (not shown) disposed on a proximal portion of the shaft and the one or more balloons 276. As a result, after the bone has been reamed, the operator may inflate balloon 276 to a radially expanded condition and retract shaft 202 in the proximal direction to extract the bone and/or cellular material from the intramedullary canal. Alternatively, deformable section 214 may be formed of a sponge-like material that is designed to expand upon contacting blood. In some examples, the sponge-like material may have a porous structure designed to retain bone graft. In this regard, outer tube 204 may be disposed about the sponge during a reaming procedure to retain the sponge in a radially collapsed condition. After the bone has been reamed, outer tube 204 may be retracted in a proximal direction to expose the sponge to blood, allowing the sponge to expand and retain bone graft therein, before the shaft is retracted from the intramedullary canal.

Deformable section 214, as shown in FIG. 20, may be in the form of a flexure 278 in the shape of an inwardly bent hook. In this example, flexure 278 may be attached to a distal end of outer tube 204 via a hinge 280. Shaft 202 may include outwardly bowed arms 282. Thus, after a reaming procedure, outer tube 204 may be retracted in a proximal direction until bowed arms 282 engage the hook of flexure 278 which, in turn, causes the flexure 278 to pivot about hinge 280 and expand in a radial direction.

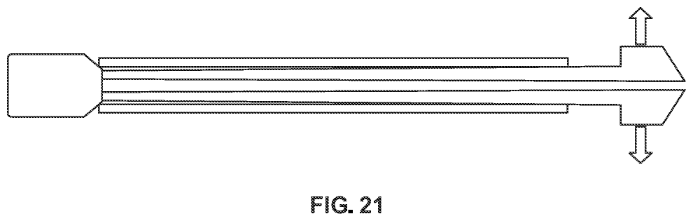

Turning to FIG. 21, deformable section 214 may be an expandable reamer head 210. As shown in FIG. 21, reamer head 210 may be a split reamer head including a first portion 284 and a second portion 286 capable of moving relative to one another. During a reaming procedure reamer head 210 may be in a collapsed condition. That is, first portion 284 and second portion 286 define a cannulation sized to receive a k-wire. After the bone has been reamed, the operator may further separate the first portion 284 and the second portion 286, for example, by actuating a trigger (not shown). In this regard, bone and/or cellular material located proximal to the reamer head 210 may be extracted as shaft 202 is withdrawn from the intramedullary canal.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A harvesting device, comprising:
an adaptor defining an internal cavity;
a storage container coupled to the adaptor and in fluid communication with the internal cavity for receiving bone and/or cellular material extracted from a patient; and
a reamer assembly at least partially disposed within the adaptor, the reamer assembly comprising:
a shaft defining a distal end arranged to receive a reamer head;
an outer tube at least partially surrounding the shaft; and
a deformable section having an elongated condition during a reaming procedure and a radially expanded condition for extracting the bone and/or cellular material from the patient,
wherein movement of the outer tube relative to the shaft in a distal direction transitions the deformable section from the elongated condition to the radially expanded condition, and movement of the outer tube relative to the shaft in a proximal direction transitions the deformable section from the radially expanded condition to the elongated condition.

2. (canceled)

3. (canceled)

4. The device of claim 1, wherein the reamer assembly is retractable relative to the adaptor.

5. The device of claim 1, wherein the deformable section comprises a resilient material.

6. The device of claim 5, wherein the resilient material is a biocompatible braided material.

7. The device of claim 1, wherein the outer tube includes a handle provided with a locking mechanism arranged to secure the outer tube at a fixed position along a length of the shaft when the deformable section is in the elongated condition and/or the radially expanded condition.

8. The device of claim 1, wherein the adaptor includes an aspiration fitting arranged to secure a tubing, and the aspiration fitting is arranged to rotate about a longitudinal axis of the adaptor.

9. The device of claim 1, wherein the adaptor comprises a detachable protective sleeve, the protective sleeve including at least one protrusion on an exterior surface thereof for securing the protective sleeve within an entry portal in bone.

10. A reamer assembly for extraction of bone and/or cellular material from a patient, the reamer assembly, comprising:
a shaft defining a distal end arranged to receive a reamer head; and
an outer tube at least partially surrounding the shaft and coupled to the shaft, the outer tube having a deformable section moveable between an elongated condition during reaming and a radially expanded condition for extracting the bone and/or cellular material from the patient,
wherein movement of the outer tube relative to the shaft in a distal direction transitions the deformable section from the elongated condition to the radially expanded condition, and movement of the outer tube relative to the shaft in a proximal direction transitions the deformable section from the radially expanded condition to the elongated condition.

11. The reamer assembly of claim 10, further comprising the reamer head and wherein the reamer head is removably attachable to the distal end of the shaft.

12. The reamer assembly of claim 11, wherein the reamer head and the distal end of the shaft are removably secured via a dove-tail type coupling.

13. The reamer assembly of claim 10, wherein the shaft is permitted to rotate freely within the outer tube.

14. (canceled)

15. The reamer assembly of claim 10, wherein a cross-section of the deformable section has a first diameter in the elongated condition and a second diameter in the radially expanded condition, and wherein the second diameter is larger than the first diameter.

16. A method of collecting bone and/or cellular material during a harvesting procedure, the method, comprising:
(a) reaming a canal of a bone to generate bone and/or cellular material using a harvesting device including a shaft, an outer tube at least partially surrounding the shaft and a deformable section;
(b) moving the outer tube relative to the shaft in a distal direction to transition the deformable section of a reamer assembly from an elongated condition to a radially expanded condition;
(c) after the moving step, retracting the reamer assembly to mechanically extract the bone and/or cellular material from a patient; and
(d) collecting the bone and/or cellular material in a storage container.

17. The method of claim 16, further comprising the step of moving the outer tube relative to a the shaft of the reamer assembly in a proximal direction to transition the deformable section from the radially expanded condition to the elongated condition.

18. The method of claim 16, further comprising:
(e) removing a first reamer head from the reamer assembly after performing step (d);
(f) securing a second reamer head to the reamer assembly after performing step (e), the second reamer head being larger than the first reamer head; and
(g) reaming the canal of the bone to generate additional bone and/or cellular material using the harvesting device.

19. The method of claim 16, wherein the bone is one of a femur or a tibia bone.

20. The method of claim 16, wherein the reaming step (a) includes reaming a counterbore within an entry portal of the bone, and the method further comprises securing a protective sleeve of an adaptor within the reamed counterbore via an interference fit.

21. The device of claim 1, wherein the adaptor includes an aspiration fitting arranged to secure a tubing, and the aspiration fitting is arranged to pivot about a connection point.

22. The device of claim 1, further comprising an end cap at a proximal end of the device, the end cap comprising a slit configured to receive the reamer assembly when the reamer assembly is retracted.

23. The method of claim 16, wherein the retracting step (c) includes receiving the reamer assembly through a slit in an end cap at a proximal end of the device so that the proximal end is sealed.
